(19) United States
(12) Patent Application Publication
(10) Pub. No.: US 2025/0222193 A1
(43) Pub. Date: Jul. 10, 2025

(54) PRESSURE MONITORFNG IN IRRIGATION SYSTEM

(71) Applicant: Coloplast A/S, Humlebaek (DK)

(72) Inventors: Niels Hvid, Vedbaek (DK); Benjamin Holm Salka, Copenhagen N (DK)

(21) Appl. No.: 18/853,445

(22) PCT Filed: Mar. 29, 2023
(86) PCT No.: PCT/DK2023/050067

§ 371 Date: Oct. 2, 2024

(30) Foreign Application Priority Data
Apr. 8, 2022 (DK) PA202270188

(51) Int. Cl. A61M 3/02

(52) U.S. Cl. CPC A61M 3/0216; A61M 3/0202; A61M 3/0233; A61M 2205/07; A61M 2205/3331; A61M 2205/50; A61M 2210/1064

(57) ABSTRACT

An irrigation system is disclosed, the system comprising a housing comprising a reservoir for containing an irrigation liquid, and an electronics compartment sealed from the reservoir; a tubing providing fluid communication between the reservoir and a catheter for use with the irrigation system; a control unit comprising at least one processor; a pump for pressurizing the reservoir to facilitate a flow of irrigation liquid from the reservoir, through the tubing, to the catheter, the pump being controllable by the at least one processor; an absolute pressure sensor in communication with the at least one processor and configured to measure the absolute pressure in the reservoir; and a relative pressure sensor in communication with the at least one processor and configured to measure the relative pressure between the reservoir and the electronics compartment. The at least one processor is configured to control the irrigation procedure in accordance with the absolute pressure and the relative pressure.

The present disclosure relates to an irrigation system for irrigation of the bowels of a user, the system comprising an absolute pressure sensor and a relative pressure sensor for control of the irrigation procedure. Further, the disclosure relates to a method of controlling an irrigation procedure.

### BACKGROUND

Bowel irrigation is one of a number of treatments used to aid people with bowel problems. People suffering from bowel problems are often paralyzed, typically due to spinal cord injuries, and confined to a wheelchair or hospitalized. In these situations, often the peristaltic functions, i.e. the reflexes and muscles of the bowel, cannot be stimulated correctly. This results in constipation or random discharge of bowel contents. By using bowel irrigation, a stimulation of the peristaltic movements of the colon can be provided. To perform such bowel irrigation, a device comprising a catheter, also referred to as an anal catheter, anal probe, rectal catheter, or speculum, is provided.

The catheter is inserted into the rectum through the anus. A liquid, also referred to as an irrigation liquid, such as water or a saline solution, is then introduced into the rectum/bowels through the catheter. The amount of liquid is generally up to 1.5 litres, depending on the person. The introduced liquid stimulates the peristaltic movements of the bowel. After a specified period of time, such as 15 minutes, the catheter is removed, and the liquid, along with output from the bowel, is released through the anus.

### BRIEF DESCRIPTION OF THE DRAWINGS

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates an exemplary irrigation system according to an embodiment of the invention;

FIG. 2 illustrates an exemplary cross-sectional view of a housing of an irrigation system according to an embodiment of the invention;

FIG. 3 illustrates an exemplary graph of the relative pressure and the absolute pressure as a function of time according to an embodiment of the invention;

FIG. 4 illustrates a method of controlling an irrigation procedure in an irrigation system according to embodiments of the invention; and

FIG. 5 illustrates a logic system for use with an irrigation system according to embodiments of the invention.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

In the following, whenever referring to a proximal end of an element of the invention, the referral is to the end adapted for insertion. Whenever referring to the distal end of an element, the referral is to the end opposite the insertion end. In other words, the proximal end is the end closest to the user, when the catheter is to be inserted and the distal end is the opposite end—the end furthest away from the user when the catheter is to be inserted. The longitudinal direction is the direction from the distal to the proximal end. The transverse direction is the direction perpendicular to the longitudinal direction, which corresponds to the direction across the shaft of the catheter.

In the following, whenever referring to a bowel irrigation system or bowel irrigation, the referral to a system or method capable of irrigating the bowels of a user using a catheter. Commonly, the catheter is inserted through the anus. Bowel irrigation (systems) is also known in the art as anal irrigation (systems) and rectal irrigation (systems), and the terms may be used interchangeably in embodiments where the bowel irrigation system is adapted for use through the anus.

In the following, whenever referring to the bowel(s) of a user, the referral is to the intestines of the user. The referral can be to the lower intestines specifically, e.g., the rectum and/or the colon/large intestine. In the following, whenever referring to the rectum, the referral is to the terminal section/canal of the intestine ending in the anus. In the following, whenever referring to the anus, the referral is to the opening of the lower end of the alimentary canal, through which refuse of digestion is commonly excreted. In the following, whenever referring to anal, the referral is to a feature, device, method, or system pertaining to the anus, e.g., pertaining to engagement with or through the anus. In the following, whenever referring to the rectal walls, the referral is to the intestinal wall surrounding and defining the canal of the rectum.

In the following, whenever referring to a quantity, such as pressure, being in compliance with a threshold value, the referral is to the quantity being within the range where such quantity attains a desired value. Where the threshold value defines an upper limit to an acceptable value of the quantity, compliance is fulfilled whenever the value is equal to or below the threshold value.

Likewise, where the threshold value defines a lower limit to an acceptable value of the quantity, compliance is fulfilled whenever the value is equal to or above the threshold value. Accordingly, non-compliance is used to describe the value of the quantity not being in compliance, i.e., falling outside the scope of compliance.

In the following, the following conversions may be used. 1 psi=7×10¹ mbar=7 kPa, and 1.0 psi=69 mbar=6.9 kPa, and 1.00 psi=68.9 mbar=68.9 kPa, and 1.000 psi=68.95 mbar=6.895 kPa. Further, 1.000 atm=14.70 psi=1013 mbar=101.3 kPa.

In the following, whenever referring to a fluid, the referral is to any liquid, gas or other material that continuously deforms under an applied shear stress, or external force, as defined in fluid dynamics. In particular, within the present invention, the term “fluid” includes both the liquid and gas phases. In other words, a fluid may be a liquid or a gas. In the following, whenever referring to a liquid, the referral is to a (nearly) incompressible fluid that conforms to the shape of its container but retains a (nearly) constant volume independent of pressure. In the following, whenever referring to a gas, the referral is to a fluid that has neither independent shape nor volume. In particular, within the present invention, the gas may be (ambient) air, unless otherwise specified. Thus, “gas” and “air” may be used interchangeably.

The present disclosure provides an irrigation system and a method of controlling an irrigation procedure in, or a pump of, an irrigation system.

In a first aspect of the invention, an irrigation system is disclosed. The irrigation system is for running an irrigation procedure for irrigation of the bowels of a user. In other words, the irrigation system is for irrigation of the bowels of the user, and in particular for irrigation of the bowels of a user via employment of an irrigation procedure as provided by the system. The irrigation system comprises:
a housing comprising a reservoir for containing an irrigation liquid, and an electronics compartment sealed from the reservoir;
a tubing providing fluid communication between the reservoir and a catheter for use with the irrigation system;
a control unit comprising at least one processor;
a pump for pressurizing the reservoir to facilitate a flow of irrigation liquid from the reservoir, through the tubing, to the catheter, the pump being controllable by the at least one processor;
an absolute pressure sensor in communication with the at least one processor and configured to measure the absolute pressure in the reservoir; and
a relative pressure sensor in communication with the at least one processor and configured to measure the relative pressure between the reservoir and the electronics compartment.

The at least one processor may be configured to control the irrigation procedure in accordance with the absolute pressure and the relative pressure. The at least one processor may be configured to control the pump in accordance with the absolute pressure and the relative pressure. In an embodiment, to control the irrigation procedure comprises to control the pump, such as the operation of the pump. In the following, a reference to “control the irrigation procedure” may be a reference to “control the pump”. The reference “to control the irrigation procedure” is used for simplicity, but it is appreciated that an irrigation procedure may be performed even without association with a human body. In particular, it is appreciated that “to control the irrigation procedure” may be embodied by controlling the pump, or another element or feature of the irrigation system.

The system may comprise a catheter for arrangement in a user. The catheter may be coupled, such as releasably coupled, to an end of the tubing.

The irrigation system may be said to be configured to run or provide an irrigation procedure, whereby it is meant that the irrigation system is configured to, by means of the features of the system as disclosed, to irrigate the bowels of a user, said irrigation of the bowels of the user being the irrigation procedure.

The irrigation system may be a portable irrigation system. By a portable irrigation system is meant a system having a size and weight suitable for carry by a user, such that the user may use the system in various settings and in various places. For example, the irrigation system may have means for operating without external power, such as by means of a (rechargeable) battery/power unit. In embodiments, the battery is rechargeable via a charging interface of the housing of the irrigation system. In an alternative embodiment, the irrigation system is powered by means of a wired connection to an outlet of a power grid.

The housing comprises a reservoir. The reservoir may be any reservoir suitable for holding a liquid. In embodiments, the reservoir can be considered a container. In embodiments, the reservoir is capable of storing at least the amount of liquid required for an irrigation procedure. The reservoir is sealable, such as to avoid spillage. In embodiments, the reservoir is hermetically sealable such that fluids may not escape the reservoir through other openings than that of the tubing.

In embodiments, the irrigation liquid is water, such as tap water, or a saline solution. In embodiments, the provision of liquid can be through a simple procedure of gaining access to the reservoir, e.g., by removing a lid of the housing, and pouring liquid into the reservoir.

The housing comprises an electronics compartment sealed from the reservoir. The electronics compartment may comprise electronics associated with the irrigation system, such as a powering circuit, including a power unit/battery, the control unit, and the pump. In an embodiment, the electronics compartment comprises at least parts of the pump, at least parts of the control unit, and at least parts of a powering circuitry configured to power the irrigation system. The electronics compartment may further comprise additional elements cooperating with, or connected to, parts of the powering circuit, the control unit, and/or the pump. In an embodiment, the electronics compartment is vented relative to surroundings. In a preferred embodiment, the electronics compartment is vented via a filter, the filter preventing liquids and/or particles from entering the electronics compartment. The filter may comprise or introduce a certain resistance to air entering or escaping the electronics compartment. The electronics compartment is preferably sealed relative to the reservoir such that liquid or gasses from the reservoir cannot enter the electronics compartment. The electronics compartment may comprise one or more sensors connected to at least the at least one processor. The one or more sensors may comprise the absolute and relative pressure sensors as disclosed, which will be discussed in greater detail below.

In embodiments, the irrigation system comprises a catheter. A catheter may also be denoted an anal probe in the art. In embodiments the catheter is provided with eyelets in the proximal end, the eyelets communicating with an irrigation channel inside the catheter, so that irrigation liquid pumped into the catheter in a distal end can exit the catheter through the eyelets in the proximal end. Tests have shown that a diameter of the irrigation channel of approximately 3-7 mm, for example 4.3 mm, allows an adequate flow. The catheter may comprise retention means for securely arranging the catheter in the anus of the user. Retention means may comprise an inflatable balloon.

In embodiments, the catheter is adapted for insertion into a rectum of the user and for arrangement in the user. One useful exemplary catheter comprises a main tubular part, typically called a shaft, extending from the distal end to the proximal end. A tip is positioned in the proximal end of the catheter and is provided as a rounded closed end of the shaft. In embodiments, the catheter comprises a connector in the distal end and may in an embodiment comprise a flared end of the catheter so that the diameter of the connector increases with respect to the tubular part. In particular, the proximal end of the catheter is configured for insertion into the rectum of the user. Usually, catheters used for bowel irrigation are 8-16 mm in external diameter, for example 10 mm. The length can be 70-200 mm, for example 150 mm. In embodiments, the catheter is of a size reflecting the needs or requirements of the user. As such, a range of different catheter sizes can be provided.

In embodiments, the catheter is adapted for insertion by means of having an appropriate size, shape, and through an appropriate material choice. In embodiments, a method of bowel irrigation comprises the step of inserting the catheter into a rectum of a user. In embodiments, insertion of the catheter into the rectum is carried out manually by the user or by a health care professional. In embodiments, insertion is aided by the provision of a lubricant. In embodiments, insertion is aided by a certain surface treatment of the catheter reducing its friction. In embodiments, the catheter is inserted by a distance such that at least the proximal end with eyelets is past an anal sphincter of the user, such as the internal anal sphincter.

The irrigation system may comprise a tubing providing fluid communication between the reservoir and a catheter for use with the irrigation system. In embodiments the provision of a tubing connecting the reservoir and a catheter facilitates transfer of liquid from the reservoir to the rectum via eyelets in the proximal end of the catheter and therefrom into the rectum once inserted. In embodiments, the tubing comprises at least one lumen extending from a first end to a second end of the tubing. In embodiments, the first end of the tubing extends into the reservoir, or is arranged in communication with the reservoir, e.g., via a channel in the housing of the irrigation system, such that irrigation liquid contained in the reservoir may enter the tubing via the first end. In particular, in embodiments, the first end of the tubing may extend to, or be arranged within or in proximity to, a bottom of the reservoir. Here, the bottom of the reservoir denotes the part of the reservoir where liquid will accumulate when the housing is arranged on a plane surface and exposed to gravity. In other words, the first end of the tubing may be configured to extend into the irrigation liquid when such liquid is arranged/contained in the reservoir. The first end may form part of a dip-tube configured to extend into the irrigation liquid when such liquid is arranged/contained in the reservoir. In embodiments (as discussed later), where the pump is an air pump configured to pressurize the reservoir, such pressurization may cause liquid to be forced into the tubing due to the provision of the dip tube or tubing extending into the irrigation liquid.

In embodiments, the second end of the tubing comprises a connector configured to connect/mate with a catheter. In embodiments where retention means of the catheter comprises a balloon, the tubing may comprise two lumens including a first and a second lumen, the first lumen connecting the interior of the balloon and the reservoir, and the second lumen connecting the tip of the catheter and the reservoir. In embodiments, the tubing is flexible. In embodiments, at least parts of the tubing are transparent for providing visual assessment of the flow of fluid.

In embodiments, the tubing is integral from the first end to the second end. In a preferred embodiment, the tubing comprises two or more segments including a dip tube arranged in the reservoir, a flexible tubing segment extending from the housing to the catheter, and a channel or intermediate tube section/segment connecting the dip tube and the flexible tubing segment. For example, the intermediate tube section may be a channel provided in the housing and comprising a connector for connecting the flexible tubing segment to the housing and thus the dip tube.

In embodiments, a first part of the tubing is flexible, and a second part of the tubing is rigid. In embodiments, the first part extends from the housing to the second end of the tubing, and the second part is formed as a channel in, or integrated part of, the housing. In embodiments, the dip-tube is flexible. In embodiments, the dip-tube is rigid. In embodiments, the tubing comprises the dip tube, the intermediate tube section, and a flexible tubing comprising a connector for connecting to a catheter. The intermediate tube section may be arranged in the lid such that fluids may enter the dip tube and flow through said dip tube, through the intermediate tube section of the lid and further into the flexible tubing connected to a catheter. Thus, by “tubing” is meant a pathway or lumen providing fluid communication between the first end and the second end, and such pathway or lumen may be partly rigid and formed as part of the housing, and partly flexible and extending out of the housing. In other words, when a tubing is discussed in the following, the reference is to a tubing providing fluid communication between the reservoir and a catheter for use with the irrigation system, and it is appreciated that such tubing may comprise multiple elements which, when assembled, provides such fluid communication through a lumen.

The irrigation system comprises a control unit. The control unit may be built into the housing, such as contained in a part of the housing separate from the reservoir, such as in an electronics compartment. In a preferred embodiment, the control unit is contained in a lid for the reservoir. The control unit comprises at least one processor. The control unit may comprise a memory connected to the processor and an interface allowing connection to the at least one processor. The control unit may comprise one or more buttons including a power button for turning the irrigation system on/off. The control unit may comprise a button for turning the pump on/off.

In an embodiment, the control unit comprises a user interface. For example, the user interface may be for receiving inputs from the user or providing outputs to the user. For example, the user interface may be a graphical user interface for presenting visual information to the user. The interface may be provided in a surface of the lid of the irrigation system where the control unit is provided in such lid. The control unit may comprise one or more indicators, such as light indicators, such as LED's, for communicating an operating status of the irrigation system. The control unit may comprise means for providing an audible signal. In embodiments, the audible signal is provided by adequate control of the pump for generating a sound originating from the pump mechanics. The control unit may comprise means for providing a haptic feedback.

In embodiments, the user interface is provided in a remote, such as a handheld remote configured to communicate with an interface and a processor of the irrigation system via wired or wireless means. The remote may comprise a second processor and/or memory. The remote may be an accessory control unit configured to communicate with a control unit built into the housing.

In embodiments, an electrical wiring is provided within the electronics compartment to provide an electrical connection between the pump and the control unit, such that the control unit, and in particular the at least one processor thereof, can be used to control the performance of the pump and/or other parts of the irrigation system. In embodiments, the control unit and the pump are in communication according to a wireless protocol.

The irrigation system comprises a pump for pressurizing the reservoir to facilitate a flow of irrigation liquid from the reservoir, through the tubing, to the catheter. The pump is controllable by the at least one processor.

In a preferred embodiment, the pump is an electrical air pump. In embodiments, the pump is powered by a power unit of the irrigation system, such as via a battery contained in the housing, such as in the electronics compartment. The power source provides an operating voltage to the pump. The power source may further power further electronics of the system, such as the processor(s) and sensors. In embodiments, the pump is arranged in the electronics compartment and comprises an air inlet and an air outlet. The air inlet may be an opening towards the surroundings for taking in ambient air. The air outlet may be an opening towards the reservoir for pumping in, and thus increasing the air pressure in, the reservoir. The air inlet and/or air outlet may be provided with an air filter.

The pump may facilitate a flow of fluid in a tubing. In a preferred embodiment, the pump is configured to pressurize the reservoir by means of increasing the air pressure inside the reservoir. For example, the pump may be an electrical air pump configured for pumping air into the reservoir by taking in ambient air. In a preferred embodiment, the reservoir is hermetically sealable. In a preferred embodiment, the pressurization of the reservoir causes liquid to escape the reservoir via tubing in communication with the reservoir. In particular, the tubing should be arranged such that its first end is covered with liquid. This may be achieved by means of a dip tube of the tubing extending into the liquid in the reservoir during normal use. For example, where the housing, and thus the reservoir, is provided with a preferred bottom upon which the user is expected to place the housing on a plane surface, the first end of the tubing may be arranged near this bottom. Thereby, when the irrigation system is prepared for irrigation, liquid will cover the first end/entrance of the dip tube and thus the tubing, and when the air pressure inside the reservoir is increased, the liquid will be forced into the tubing and, for example, further into the rectum when a catheter connected to the tubing is inserted in the rectum of a user.

The irrigation system comprises an absolute pressure sensor in communication with the at least one processor and configured to measure the absolute pressure in the reservoir. For example, the processor may receive absolute pressure data indicative of the absolute pressure in the reservoir from the absolute pressure sensor. By an absolute pressure sensor is meant a pressure sensor configured to measure the air pressure relative to vacuum, also denoted the absolute pressure. Thereby, under normal circumstances at sea level (Normal Temperature and Pressure (NTP)), the air pressure will read one standard pressure; 1 atm=1013.25 mbar=1013.25 hPa. For example, the absolute pressure sensor may be a commercially available absolute pressure sensor comprising a vacuum portion and a membrane deflectable in response to an exerted pressure, where the vacuum portion provides for measuring the pressure relative to zero, being the definition of absolute pressure.

The irrigation system comprises a relative pressure sensor in communication with the at least one processor and configured to measure the relative pressure between the reservoir and the electronics compartment. For example, the processor may receive relative pressure data indicative of the relative pressure between the reservoir and the electronics compartment from the relative pressure sensor. By a relative pressure sensor is meant a pressure sensor configured to measure the relative pressure between two compartments (e.g., the reservoir and the electronics compartment). Thus, the relative pressure may be either negative or positive, dependent on the point of view, as the pressure in a first compartment (e.g., the reservoir) is now measured relative to the pressure in a second compartment (e.g., the electronics compartment), which may have a higher or lower pressure than the first compartment. For example, the relative pressure sensor may be a commercially available relative pressure sensor comprising a membrane deflectable in response to an exerted pressure, the direction and size of deflection being indicative of the relative pressure between the two compartments.

The pressure sensors may be configured to continuously measure the corresponding air pressure, such as at a frequency higher than 0.1 Hz, such as higher than 1 Hz, such as higher than 10 Hz or higher than 100 Hz.

The at least one processor may be configured to control the irrigation procedure in accordance with the absolute pressure and the relative pressure. The at least one processor may be configured to control the pump in accordance with the absolute pressure and the relative pressure. By control is meant that the at least one processor may be configured to influence, regulate, or affect the irrigation procedure in accordance with the absolute pressure and the relative pressure. For example, in other words, the processor may take the absolute and relative pressures, such as the respective absolute pressure data and relative pressure data, as input and generate an output comprising instructions on how to operate the pump based on said pressures/pressure data. Thus, it is appreciated that to control the irrigation procedure may be embodied by controlling features of the system, such as by controlling the pump. For example, the output may comprise instructions to power off the pump in accordance with/based on the absolute and/or relative pressure. To control may comprise one or more of to terminate, initiate, pause, resume/reinitiate the irrigation procedure (e.g., the pump) or to disable the irrigation system as such.

In embodiments, the at least one processor is configured to control the irrigation procedure in accordance with the absolute pressure and the relative pressure being in compliance (agreement) or non-compliance (disagreement) with one or more threshold values. For example, a first threshold value may be a threshold value representing a deviation or divergence between the absolute pressure and the relative pressure. In other words, the at least one processor may be configured to control the irrigation procedure in accordance with the absolute pressure and the relative pressure being in compliance (or, correspondingly, non-compliance) with a first threshold value.

An irrigation system as disclosed comprising both an absolute pressure sensor and a relative pressure sensor and using such absolute pressure and relative pressure as inputs to the control of the irrigation procedure, provides a safety mechanism configured to detect malfunctioning during an irrigation procedure. For example, malfunctioning may occur due to improper handling of the irrigation system, in particular the housing thereof, causing damage to the system, or due to wear.

Specifically, the provision of both an absolute and a relative pressure sensor solves the problem of how to reliably detect damage, such as a breach, of an interface between the reservoir and the electronics compartment, which would greatly influence the monitoring of the amount of irrigation liquid transferred to the rectum of the user during the irrigation procedure and thereby constitute a risk to the user.

As an example, the mere provision of an absolute pressure sensor configured to measure the absolute pressure in the reservoir would not necessarily successfully/reliably detect breach of an interface between the reservoir and the electronics compartment. For example, the decrease in pressure in the (pressurized) reservoir because of such breach may be negligible or originate from other mechanisms, including the expected decrease in pressure resulting from the reservoir being emptied during the irrigation procedure, such as if the pump is turned off. Further, by considering solely the absolute pressure, one cannot determine whether a lack of increase in pressure is due to air escaping the reservoir via an improperly closed lid or via a damaged interface between the reservoir and the electronics compartment, the latter being considered a more severe situation than the former.

Likewise, as an example, the mere provision of a relative pressure sensor configured to measure the relative pressure between the reservoir and the electronics compartment would not necessarily successfully/reliably detect a breach of an interface between the reservoir and the electronics compartment. As an example, whereas equilibrium in the pressures would occur eventually in case of a breach, the size of the breach would affect the time it takes such equilibrium to occur, and during such time, the decrease in relative pressure may not immediately be indicative of damage.

Finally, if using just a single pressure sensor in the system, it is a possibility that such single pressure sensor itself may be malfunctioning or damaged, whereby it would be impossible to determine any irregularities or malfunctioning of the irrigation system as such during the irrigation procedure.

On the contrary, when considering the absolute pressure and the relative pressure simultaneously, a divergence between the absolute pressure and the relative pressure provides a more failsafe indicator for malfunctioning. For example, under normal conditions, it would be expected that the absolute pressure and the relative pressure increase in parallel/agreement (albeit the zero-point may be different, or the sensors may be tared, as explained later), whereas non-agreement between the absolute pressure and the relative pressure may provide an indicator for a malfunctioning element which could require the pump (as controlled by the at least one processor) to terminate the pressurization (i.e., turn off the pump) to avoid further increase in pressure.

Thus, the provision of both an absolute pressure sensor and a relative pressure sensor provides a failsafe safety mechanism configured to detect malfunctioning of the irrigation system. In particular, the mechanism is specifically optimised to detect and prevent situations that may be considered unsafe for the user.

In an embodiment, the processor is configured to monitor agreement between the absolute pressure and the relative pressure during the irrigation procedure. It is appreciated that by the processor being configured to monitor agreement between the absolute pressure and the relative pressure, the processor is likewise configured to monitor for any disagreement between the absolute pressure and the relative pressure during the irrigation procedure. In other words, during the irrigation procedure, the processor is configured to monitor the absolute pressure and the relative pressure and determine whether said pressures are in agreement, e.g., within a range bound by an upper and lower threshold value, or whether said pressures are in disagreement, e.g., outside a range bound by the upper and lower threshold value.

In particular, as explained above, agreement may be indicative of the irrigation system working as expected, whereas disagreement may be indicative of a malfunctioning of the irrigation system. The processor may be configured to control the irrigation procedure in accordance with such agreement or disagreement and thus take different action dependent on whether the pressures are in agreement or disagreement. In embodiments, the processor is configured to continue the irrigation procedure in accordance with the absolute pressure and the relative pressure being in agreement (e.g., not interfere with the process as originally initiated).

In an embodiment, the processor is configured to terminate the irrigation procedure in accordance with the absolute pressure and the relative pressure being in disagreement. To terminate may mean to pause the pump and/or the irrigation procedure, e.g., by entering an idle mode wherein the pump/system is ready for further irrigation once the cause for disagreement is remedied, or to terminate may mean to stop the irrigation procedure and inhibit further irrigation, such as by inhibiting the pump from pressurizing the reservoir for a certain amount of time, such as for 30 minutes or more.

In an embodiment, the processor is configured to disable the irrigation system in response to a disagreement between the absolute pressure and the relative pressure. In the present context, to disable means to make the irrigation system unusable for any future irrigation procedures. In other words, to disable the irrigation system means to permanently disable the system. To disable the system may be considered a permanent termination of the irrigation procedure and as such, in embodiments, to terminate the irrigation procedure may comprise to disable the system as such. Thus, when a reference to a termination of the irrigation procedure is used, the termination may include to disable the system.

In other words, the processor may comprise a kill-switch algorithm which, when employed, causes the irrigation system to be disabled. This functionality may be particularly relevant where a disagreement between the absolute pressure and the relative pressure is indicative of a structural malfunctioning of the irrigation system (e.g., due to damage on the system), whereby future irrigation procedures by means of the same irrigation system are not recommended. For example, the user may thus be requested to discard of or return the system to a manufacturer.

In embodiments, the system may run for a certain time-period following a determination of disagreement before the system is disabled. During such time-period, the processor may analyse the pressures and confirm whether the disagreement is indeed indicative of malfunctioning, or whether the disagreement is caused by minor fluctuations. The time-period may, for example, be selected between 1 s and 30 s, such as between 10 s and 30 s, such as 10 s, 20 s, or 30 s.

In an embodiment, the absolute pressure sensor and the relative pressure are configured to be tared. By tared is meant that the absolute pressure sensor and the relative pressure sensor may be zeroed, such that, from the time of being tared and onwards, the absolute pressure and the relative pressure are coinciding. For example, taring the pressure sensors may be useful to disregard local variations in the absolute pressure (e.g., as caused by a change in altitude or weather) and simplify the algorithm used to monitor for agreement/disagreement.

In an embodiment, the absolute pressure sensor and the relative pressure sensor are configured to be tared in response to an initiation of the irrigation procedure. For example, the pressure sensors may be tared automatically when the user turns on the irrigation system and before the pump is turned on. Thereby, the taring is up to date and pertinent to the present irrigation procedure.

The tared pressures may be used in the assessment of agreement/disagreement as monitored by said processor: namely, when the pressures are tared, agreement between the absolute pressure and the relative pressure may be visualized by the pressures being coinciding (distance between the pressures is 0 Pa). In other words, the processor may be configured to monitor any deviation between the tared pressures, such deviation thus being indicative of disagreement.

Taring the pressures may be a matter of offsetting one pressure relative to the other. For example, in a preferred embodiment, an algorithm of the processor is configured to define a first pressure P1 and a second pressure P2 at a calibration time t_{c} being prior to turning on the pump (i.e., when the relative pressure is zero), where:

$$P 1 ( t ) = {P}_{Rel} ( t )$$

$$P 2 ( t ) = {P}_{Abs} ( t ) - Δ Po$$

Here, P_{Rel}(t) is the pressure as measured by the relative pressure sensor, P_{Abs}(t) is the pressure as measured by the absolute pressure sensor, and ΔPₒ is a constant defined at time tₒ, where tₒ≤t_{c}:

$$Δ Po = {P}_{Abs} ( to ) - {P}_{Rel} ( to )$$

ΔPₒ may be considered an offset of the absolute pressure P_{Abs}(t) in the subsequent monitoring of said absolute pressure. The offset ΔPₒ, which is defined prior to each irrigation procedure (namely, at tₒ<t_{c}), serves to eliminate local environmental factors such as weather and altitude affecting the absolute pressure but not the relative pressure.

Upon calibration at t=t_{c}, as is also realised by the formula for P2(t), the offset ΔPₒ of the absolute pressure P_{Abs}(t) by the relative pressure P_{Rel}(tₒ) causes P1(t) and P2(t) to be coinciding, such that disturbances to the system are reflected by a deviation from each other. In other words, in this embodiment, “agreement” between the relative pressure (as contained in P1(t)) and the absolute pressure (as contained in P2(t)) may be defined as:

$$P 1 ( t ) = P 2 ( t )$$

Thus, in embodiments, agreement between the relative pressure and the absolute pressure means that the readings of the respective pressure sensors are identical (when the absolute pressure is offset by ΔPₒ as defined). Physically, this corresponds to the respective pressure sensors being exposed to the same conditions: the pressure as measured by the relative pressure sensor is the pressure as measured by the absolute pressure sensor (when offset by ΔPₒ), and likewise; changes in the pressure as measured by the relative pressure sensor is reflected by like changes in the pressure as measured by the absolute pressure sensor (when offset by ΔPₒ).

Likewise, and correspondingly, disagreement may be defined as:

$$P 1 ( t ) \neq P 2 ( t )$$

“Agreement” may also be denoted compliance, whereas “disagreement” may be denoted non-compliance or a situation where the relative are absolute pressure deviate from each other.

In embodiments, the condition for disagreement is fulfilled when a distance between the first pressure P1 and the second pressure P2 is greater than a primary threshold value. Likewise, in embodiments, the condition for agreement is fulfilled or maintained when a distance between the first pressure P1 and the second pressure P2 is at or below the primary threshold value.

According to embodiments of the invention, the processor is configured to monitor this agreement between the relative pressure and the absolute pressure. By being configured to monitor means that the processor may continuously consider the correspondence between P1(t) and P2(t), and upon disagreement, corresponding to the situation where P1(t); P2(t), the processor may interfere with (control) the irrigation procedure, such as terminate the irrigation procedure, disable the irrigation system, or issue a notification being indicative of such disagreement to the user.

In an embodiment, the processor is configured to communicate an error state via the user interface in response to a disagreement between the absolute pressure and the relative pressure. In embodiments, the user interface is provided through appropriate control of the pump giving off a characteristic noise/audible signal and/or vibration. In embodiments, the user interface is a light diode/LED giving off a signal, such as a blinking light or a certain colour, indicative of the error state. In embodiments, the user interface is a graphical user interface, such that the error state may be communicated by means of a text message. In embodiments, the error state is for communicating to the user that action is required, such as restarting the system or stop using the system going forward (e.g., return to manufacturer).

In an embodiment, the control unit comprises at least two processors including a first processor connected to the absolute pressure sensor a second processor connected to the relative pressure sensor, the first processor and the second processor further being interconnected by at least one digital signal line. Thereby, each pressure sensor has a dedicated processor. Further, the processors are connected, such as to communicate/share data and/or instructions.

It is appreciated that embodiments discussed above in relation to the use of at least one processor are applicable to the use of at least two processors. For example, the control of the irrigation procedure as disclosed in various embodiments may be performed by either the first or the second processor, or by the first and second processor in unison, as they are interconnected via the digital signal line.

Providing a dedicated processor to each pressure sensor provides an even more fail-safe solution to the problems solved by the present invention. Namely, the provision of a dedicated processor allows for the detection of not only damage to the system (e.g., breach of a seal), but also detection of any malfunctioning of the pressure sensors as such, and of the processors as such.

In an embodiment, the first processor is configured to receive absolute pressure data from the absolute pressure sensor and to send absolute pressure data to the second processor via the digital signal line, and the second processor is configured to receive relative pressure data from the relative pressure sensor and to send relative pressure data to the first processor via the digital signal line.

Thus, the first processor, which is connected to the absolute pressure sensor, is configured to receive absolute pressure data from this absolute pressure sensor. The absolute pressure data is indicative of the absolute pressure as measured by the absolute pressure sensor. Likewise, the second processor, which is connected to the relative pressure sensor, is configured to receive relative pressure data from this relative pressure sensor. The relative pressure data is indicative of the relative pressure as measured by the relative pressure sensor.

Further, the first processor is configured to send/share the absolute pressure data to/with the second processor, and the second processor is configured to send/share the relative pressure data to/with the first processor. Thereby, all the data received by the pressure sensors is available to each processor, and each processor may thus perform the same calculations/apply the same algorithms and control the irrigation system independently.

In an embodiment, the first processor is configured to terminate the irrigation procedure in response to (i) the first processor not receiving relative pressure data from the second processor and/or (ii) disagreement between the absolute pressure data and the relative pressure data as received from the second processor, and the second processor is configured to terminate the irrigation procedure in response to (a) the second processor not receiving absolute pressure data from the first processor and/or (b) disagreement between the relative pressure data and the absolute pressure data as received from the first processor. Thereby, both of the first processor and the second processor may terminate the irrigation procedure in accordance with the two scenarios:
I) the first (second) processor does not receive relative (absolute) pressure data from the second (first) processor,
II) disagreement between the absolute (relative) pressure and the relative (absolute) pressure data as received from the second (first) processor.

In the first scenario, not receiving pressure data from the other processor may be indicative of either the pressure sensor connected to said other processor is not working, and/or said other processor from which data was expected is not working. Either case is considered critical to the irrigation procedure and as such, the working processor may terminate the irrigation procedure, and, preferably, disable the irrigation system.

In the second scenario, disagreement between the pressure data as received directly from the connected pressure sensor and the pressure data as received from the other processor may be caused by the aforementioned disagreement P1(t); P2(t), and in which case both of the processors are expected to determine this disagreement, whereby both of the processors may terminate the procedure/disable the system. Alternatively, the second scenario may be caused by a malfunctioning pressure sensor, e.g., a pressure sensor being damaged such that it generates incorrect reading, which should also be remedied by terminating/disabling the system.

Thus, the provision of two processors as disclosed facilitates a monitoring of the functioning of the pressure sensors and the processors as such, in addition to the monitoring of disagreement of the pressures caused, e.g., by a malfunctioning system or a breach of seal in the interface between the reservoir and the electronics compartment.

In an embodiment, the first processor is connected to a first transistor via a first logic line and the second processor is connected to a second transistor via a second logic line, the first transistor further connected to a voltage source via a first voltage line and the second transistor further connected to the first transistor via a second voltage line and to the pump via a third voltage line. In the present context, by a transistor is meant a logic gate commonly employed in electrical circuits, and as such, the terms may be used interchangeably herein.

Thereby is provided a direct communication with the voltage supply to the pump: namely, each of the processors may be configured to generate a logic (binary) output, which is routed to the respective transistor of the disclosed logic system, and thereby may directly shut off the voltage supply to the pump. Thereby, in the immediate response to a disagreement for any reason discussed above, the respective processor may cause the voltage supply to the pump to be terminated, irrespective of the functioning of the other processor. It is noted that in present context, the termination of the power supply to the pump, being an air pump configured to pressurize the reservoir to facilitate a flow of liquid in the tubing, is equivalent to terminate the irrigation procedure: without sustained air pressure in the reservoir, the flow of liquid through the tubing to the rectum of the user will end.

In an embodiment, the first transistor and the second transistor are configured and AND-gates.

It is appreciated that an equivalent logic system may be realised by use of other gates, but the use of two AND-gates in series from the voltage supply to the pump provides a system wherein each of the first and second processor, irrespective of the other processors' output, may cause the irrigation system to be terminated.

It is appreciated that in the above discussion, the power supply to the pump is terminated by the appropriate design of a logic system comprising logic gates/transistors, but this may be followed by a subsequent step wherein the processor further employs a kill-switch algorithm permanently disabling the system, as previously discussed. Whereas the above example discloses the use of a logic system to control the pump, it is appreciated that a similar logic system may be used with another element of the system configured to influence/control the irrigation procedure.

In a second aspect of the invention, a method of controlling an irrigation procedure in an irrigation system according to the first aspect of the invention is disclosed. The method is performed by the at least one processor of the control unit. The reservoir of the irrigation system comprises an irrigation liquid. For example, the user has prepared the system by means of pouring an irrigation liquid into the reservoir prior to initiating the irrigation procedure. The method may comprise the steps of:
turning on the pump;
initiating the irrigation procedure;
monitoring agreement between the absolute pressure and the relative pressure; and
in accordance with the absolute pressure and the relative pressure being in agreement, continue the irrigation procedure; or
in accordance with the absolute pressure and the relative pressure being in disagreement, terminate the irrigation procedure.

Thus, the method comprises monitoring agreement between the absolute pressure and the relative pressure as discussed in embodiments of the first aspect of the invention. In particular, the method comprises either continuing the irrigation procedure (in accordance with the absolute pressure and the relative pressure being in agreement) or terminating the irrigation procedure (in accordance with the absolute pressure and the relative pressure being in disagreement). Thus, it is to be understood that the present method may run independently from human interaction.

In embodiments, the system is turned on (e.g., is in an idle mode) prior to performing the method in the processor according to the method. For example, the user may have prepared the system by pouring irrigation liquid into the reservoir and turning the system on. Turning on the system may comprise powering up the processors and sensors.

In embodiments, the method is performed by the at least one processor in response to the user turning on the system.

In embodiments, turning on the pump comprises receiving input indicative of the system being turned on. In embodiments, turning on the pump comprises receiving user input indicative of the intended action of turning on the pump, such as via the user interface as previously discussed.

In an embodiment, prior to turning on the pump, the method comprises the step of taring the absolute pressure sensor and the relative pressure sensor, such as tared according to embodiments discussed above in relation the first aspect of the invention.

The method comprises the step of initiating the irrigation procedure. In embodiments, initiating the irrigation procedure comprises initiating pressurization of the reservoir. In embodiments, this step is contained in the step of turning on the pump, such as where the turning on of the pump immediately results in pressurization of the reservoir according to embodiments. In embodiments, initiating the irrigation procedure means to initiate pressurization of the reservoir by means of the pump.

Following the initiation of the irrigation procedure, the processor continuously monitors agreement between the absolute pressure and the relative pressure. In accordance with the absolute pressure and the relative pressure being in agreement, the processor is configured not to interfere with the irrigation procedure, such that the irrigation procedure may continue. In accordance with the absolute pressure and the relative pressure being in disagreement, the processor is configured to interfere with the irrigation procedure, such that the irrigation procedure may terminate, which may include disabling the irrigation system as such.

It is appreciated that definitions, embodiments, and related effects and/or benefits of the first aspect of the invention may be considered applicable to the method according to the second aspect of the invention. For example, where embodiments of the first aspect of the invention disclose or entail steps of a method, it is appreciated that such steps of a method may be applicable to the method of the second aspect of the invention.

### DETAILED DESCRIPTION OF THE DRAWINGS

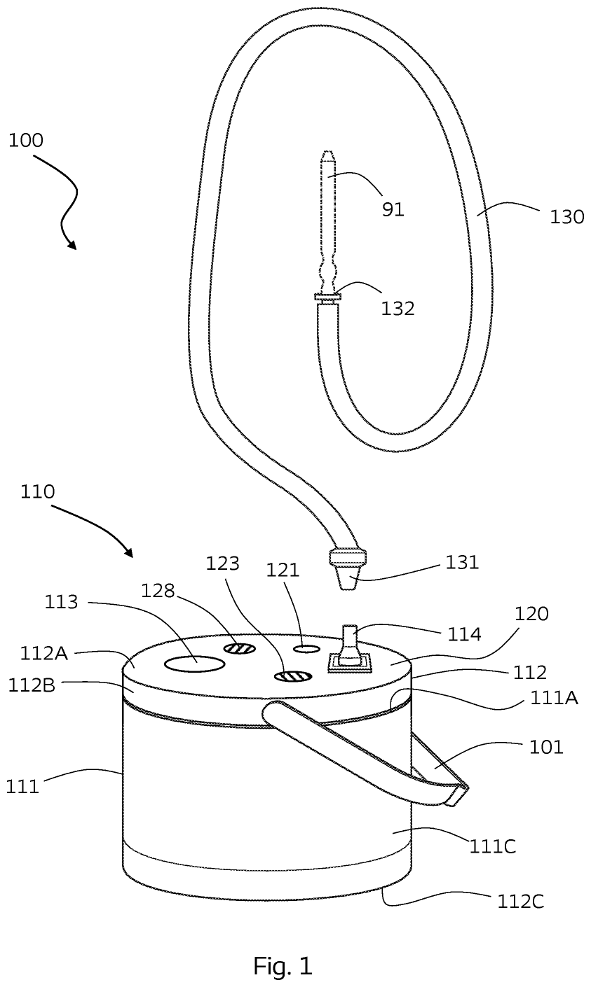

FIG. 1 illustrates an exemplary irrigation system 100 according to an embodiment of the invention. The irrigation system 100 comprises a housing 110 comprising a reservoir 111 and an electronics compartment 120 sealed from the reservoir 111. The reservoir 111 is closed off with a lid 112. In the illustrated embodiment, the electronics compartment 120 is contained in the lid 112.

The lid 112 may be detachable from the reservoir 111 to allow for filling the reservoir with an irrigation liquid and/or for easy cleaning of the interior of the reservoir. In addition, or alternatively, the lid 112 may comprise an auxiliary opening 113 providing access to the reservoir 111 through the lid. The auxiliary opening 113 may be sealable by an auxiliary lid associated with the auxiliary opening. The lid 112 is adapted to hermetically close off the reservoir 111.

The reservoir 111 may comprise a preferred bottom 111C. The preferred bottom 111C may be arranged opposite the lid 112 arranged in a top portion 111A of the reservoir 111. A wall portion 111B may extend from the preferred bottom 111C to the top portion 111A. Thus, the bottom 111C, the wall portion 111B and the top portion 111A may be said to define the reservoir 111. The preferred bottom 111C may comprise a plane surface such that the user may arrange the reservoir 111 on a table, floor or similar during irrigation on said preferred bottom.

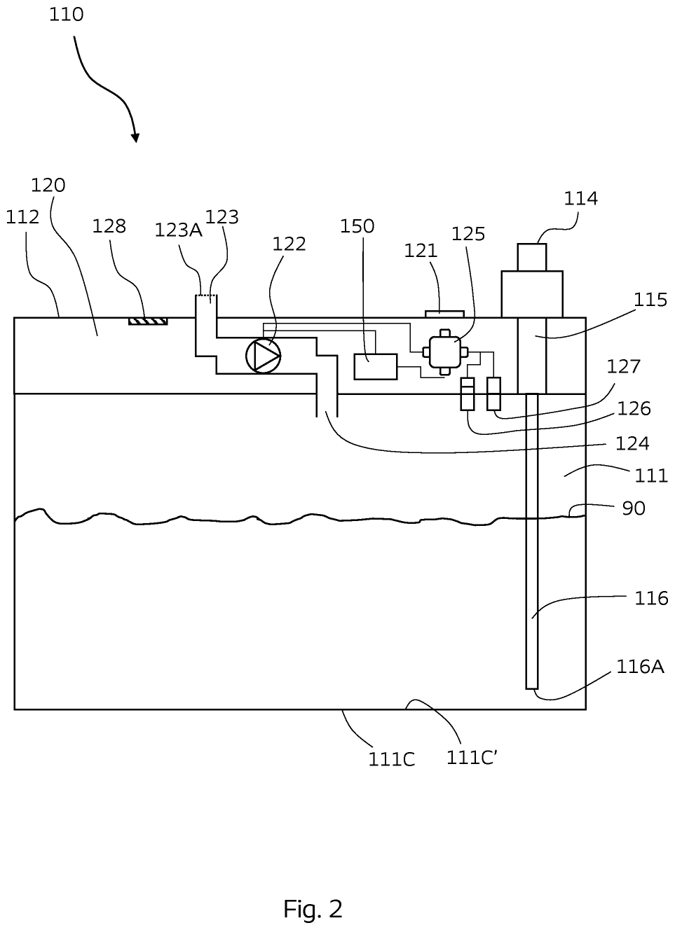

The irrigation system 100 may further comprise a tubing 130 providing fluid communication between an interior of the reservoir 111 and a catheter 90 for use with the irrigation system (see also FIG. 2 for a detailed view of the interior of the reservoir 111). The tubing 130 may be detachable from the housing 110 via a first connector 131 configured to mate with an associated tube connector 114 of the lid 112. The tubing 130 comprises a second connector 132 arranged in an opposite end of the tubing relative to the first connector 131. The second connector 132 is configured to mate with an associated connector of a catheter 91 for use with the irrigation system 100.

The irrigation system 100 may comprise a handle 101 for easy carrying. The handle 101 may be attached to the lid 113, or the handle 101 may be attached to the reservoir 111.

The irrigation system 100 comprises a control unit comprising at least one processor. In the illustrated embodiment, the control unit is built into the lid 112. The control unit may comprise an interface 121, such as a user interface for providing information to the user about the operating status of the system. For example, the interface 121 may be a light diode/LED, a graphical user interface/screen, or means for providing an audible or haptic feedback. The interface 121 may be configured to receive user input. For example, the interface 121 may be a button configured to turn the system on/off. The system may comprise one or more user interfaces 121.

The irrigation system 100 comprises an air intake 122. The air intake 123 may be arranged in the lid 112, such as in a top portion 112A or in a rim portion 112B of the lid. The air intake 123 provides ambient air for an air pump arranged in the system 100, such as in the electronics compartment 120. The pump is discussed in greater detail in relation to FIG. 2.

The electronics compartment 120 may be vented to the surroundings by means of a vent 128.

FIG. 2 illustrates an exemplary cross-sectional view of the housing 110 of an irrigation system 100 according to an embodiment of the invention. The housing 110 comprises the reservoir 111, wherein an irrigation liquid 90 is indicated, and the electronics compartment 120. In the illustrated embodiment, the electronics compartment 120 is arranged in the lid 112. The electronics compartment 120 may contain the majority of electronics associated with the irrigation system 110 including the pump 122, the at least one processor 125, and an absolute pressure sensor 126 and a relative pressure sensor 127.

The tube connector 114 of the lid 112 is in fluid communication with the reservoir 111 via an intermediate tube section 115 provided in the lid and a dip tube 116 extending into the reservoir. The dip tube 116 comprises an opening 116A wherein liquid may enter the dip tube and flow further through the tubing of the system 100. The dip tube 116 extends into close proximity of an internal bottom 111C′ of the reservoir 111 such that for a majority of the irrigation procedure, the dip tube 116 is covered with irrigation liquid 90, when the preferred bottom 111C of the reservoir 111 is arranged on a plane surface. Thereby, since the irrigation system 100 is based on an air pump 122 configured to pressurize the (air of the) reservoir 111 to facilitate a flow of irrigation liquid 90 through the tubing 130, said liquid 90 may enter the dip tube 116 via the opening 116A until said opening is not covered with irrigation liquid 90. Thus, in a preferred embodiment, the opening 116A of the dip tube 116 is arranged in close proximity of the internal bottom 111C′, or the opening 116A may be arranged within the internal bottom 111C′ via adequate arrangement of the dip tube 116.

The electronics compartment 120 may be vented relative to the surroundings via the vent 128. Preferably, the vent 128 is provided with a filter, thereby partly protecting the interior of the electronics compartment 120, and partly providing a restricted air flow between the surroundings and the electronics compartment 120.

The air pump 122 comprises an air intake 123. Preferably, the air intake 123 comprises a filter 123A. The air pump 122 comprises an air outlet 124 in communication with the interior of the reservoir 111. Thereby, the air pump 122 may pressurize the reservoir 111 by taking in ambient air via the air intake 123 and outputting the air via the air outlet 124. The pump 122 is connected to the at least one processor 125 such that the at least one processor may control the pump.

The irrigation system 100 comprises a power unit 150 coupled to the processor 125 and the pump 122. Preferably, the power unit 150 is arranged in the electronics compartment 125. The power unit 150 provides operating voltage to the processor and the pump. The power unit can be a battery, such as a rechargeable battery. The irrigation system may further comprise further electronic features not illustrated, such as means for generating, transmitting, and receiving a wireless signal, a memory coupled to the processor and further sensors.

The irrigation system 100 comprises an absolute pressure sensor 126 and a relative pressure sensor 127.

The absolute pressure sensor 126 is arranged to measure the absolute air pressure inside the reservoir 111. Thus, the absolute pressure sensor 126 may comprise an active sensor face exposed to the interior of the reservoir 111. For example, the absolute pressure sensor 126 may be a commercially available absolute pressure sensor comprising a vacuum portion and a membrane deflectable in response to an exerted pressure, where the vacuum portion provides for measuring the pressure relative to zero, being the definition of absolute pressure.

The relative pressure sensor 127 is arranged to measure the relative air pressure between the reservoir 111 and the electronics compartment 120. Thus, the relative pressure sensor 127 may comprise active sensor faces exposed to both the interior of the reservoir 111 and the interior of the electronics compartment 120. For example, the relative pressure sensor 127 may be a commercially available relative pressure sensor comprising a membrane deflectable in response to an exerted pressure, the direction and size of deflection being indicative of the relative pressure between the reservoir 111 and the electronics compartment 120.

The pressure sensors 126, 127 are connected to the at least one processor 125. Thereby, the at least one processor 125 may control the irrigation procedure in accordance with the absolute pressure and the relative pressure. For example, since the processor 125 is further connected to the pump 122, the processor may control the operation of the pump (and thus, in embodiments, the irrigation procedure) in accordance with the pressure data received from the pressure sensors 126, 127.

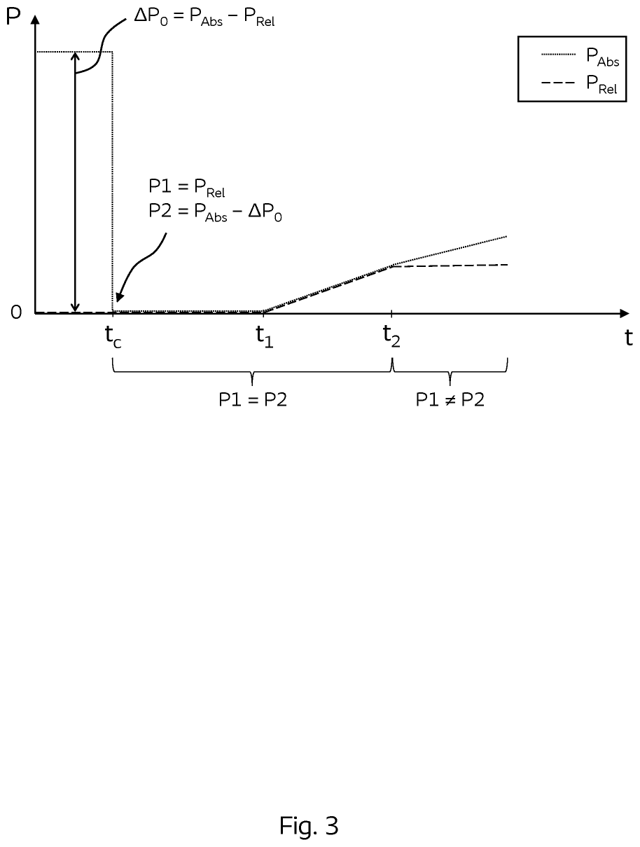

FIG. 3 illustrates an exemplary graph of the relative pressure P_{Rel} (dashed) and the absolute pressure P_{Abs} (dotted) as a function of time t according to an embodiment of the invention implementing an algorithm configured to monitor agreement between the relative pressure and the absolute pressure.

At calibration time t_{c}, a first pressure P1 and a second pressure P2 is defined as follows:

$$P 1 ( t ) = {P}_{Rel} ( t )$$

$$P 2 ( t ) = {P}_{Abs} ( t ) - Δ Po$$

where

$$Δ Po = {P}_{Abs} ( to ) - {R}_{Rel} ( to )$$

where tₒ≤t_{c}. Thus, ΔPₒ is an offset parameter defined at or prior to calibration t_{c}.

The definition of the first P1 and second pressure P2 at calibration time t_{c} may be considered a taring of the relative and absolute pressure sensors. The calibration or taring may be performed in response to an initiation of the irrigation procedure or in response to a powering on of the irrigation system as such. Thereby, the sensors are tared prior to each irrigation procedure, whereby local external variations due to altitude and/or weather are eliminated.

Practically, the definition of the second pressure P2 results in the absolute pressure P_{Abs} to be offset by the distance ΔPₒ between the absolute pressure P_{Abs} and the relative pressure P_{Rel} at the time of (or immediately before) calibration t_{c}. The first pressure P1(t) is merely indicative of the relative pressure P_{Rel}(t). Thereby, following the time of calibration t_{c}, the absolute pressure P_{Abs} (as represented by the second pressure P2(t)) and the relative pressure P_{Rel} (as presented by the first pressure P1(t)) are coinciding (as drawn as parallel dotted and dashed lines).

Following calibration, agreement exists between the first pressure P1 (indicative of the relative pressure P_{Rel} and the second pressure P2 (indicative of the absolute pressure P_{Abs}):

$$P 1 ( t ) = P 2 ( t )$$

Thus, agreement between the relative pressure Pᵣₑₗ (as represented by the first pressure P1) and the absolute pressure P_{Abs} (as represented by the second pressure P2) means that the readings of the respective pressure sensors are identical (when the absolute pressure is offset by the ΔPₒ offset as defined). Physically, this corresponds to the respective pressure sensors being exposed to the same conditions: the pressure as measured by the relative pressure sensor is the pressure as measured by the absolute pressure sensor (when offset by ΔPₒ), and likewise; changes in the pressure as measured by the relative pressure sensor are reflected by like changes in the pressure as measured by the absolute pressure sensor (when offset by ΔPₒ).

In FIG. 3, agreement is indicated between the calibration time t_{c} and a second time t₂. At a first time t₁>t_{c}, pressurization of the reservoir is initiated, whereby both the absolute pressure and the relative pressure increase in agreement. During this time, liquid may flow through the tubing to provide irrigation of the bowels of the user.

Correspondingly, disagreement may be defined as:

$$P 1 ( t ) \neq P 2 ( t )$$

In FIG. 3, disagreement is indicated following time t₂ where the absolute pressure is increasing, whereas the relative pressure is stable. This may be indicative of a pressure equalisation between the reservoir and the electronics compartment, such as due to damage to/breach of an interface between said reservoir and electronics compartment, whereby the relative pressure is zero, whereas the absolute pressure may continue to increase, such as due to a restricted airflow out of the electronics compartment (such as due to a vent configured to provide such restricted air flow between the electronics compartment and the surroundings).

“Agreement” may also be denoted compliance, whereas “disagreement” may be denoted non-compliance or a situation where the relative are absolute pressure deviate from each other.

According to embodiments of the invention, the processor is configured to monitor agreement between the relative pressure and the absolute pressure. By being configured to monitor means that the processor may continuously consider the correspondence between P1(t) and P2(t), and upon disagreement, corresponding to the situation where P1(t); P2(t), the processor may interfere with (control) the irrigation procedure, such as terminate the irrigation procedure, disable the irrigation system, or issue a notification being indicative of such disagreement to the user.

It is appreciated that the exemplary graph shown in FIG. 3 is to be considered illustrative only. For example, the increase of the respective pressures during irrigation may differ, and may be dependent on, or indicative of, the speed whereby liquid is transferred through the tubing. Likewise, the indicated period of disagreement may look different dependent on the situation. For example, the relative pressure may decrease or increase, and/or the absolute pressure may decrease or be constant. In other words, the indicated period of disagreement merely serves to illustrate one situation wherein the condition for disagreement is fulfilled, and it is appreciated that different situations may likewise fulfil the condition for disagreement.

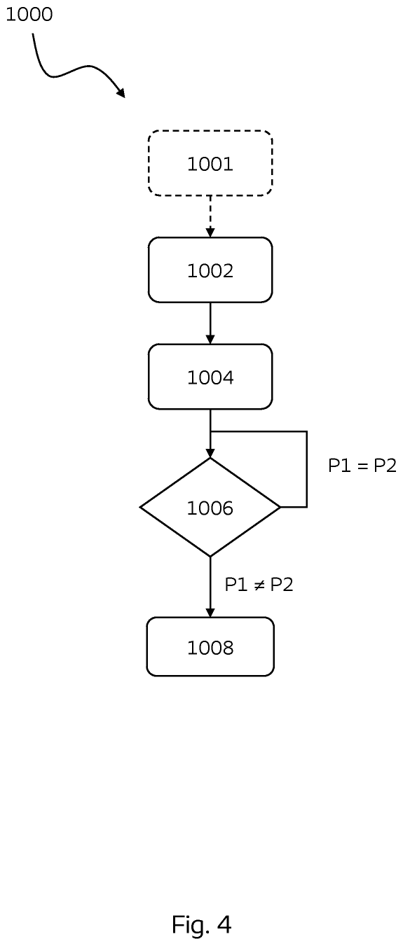

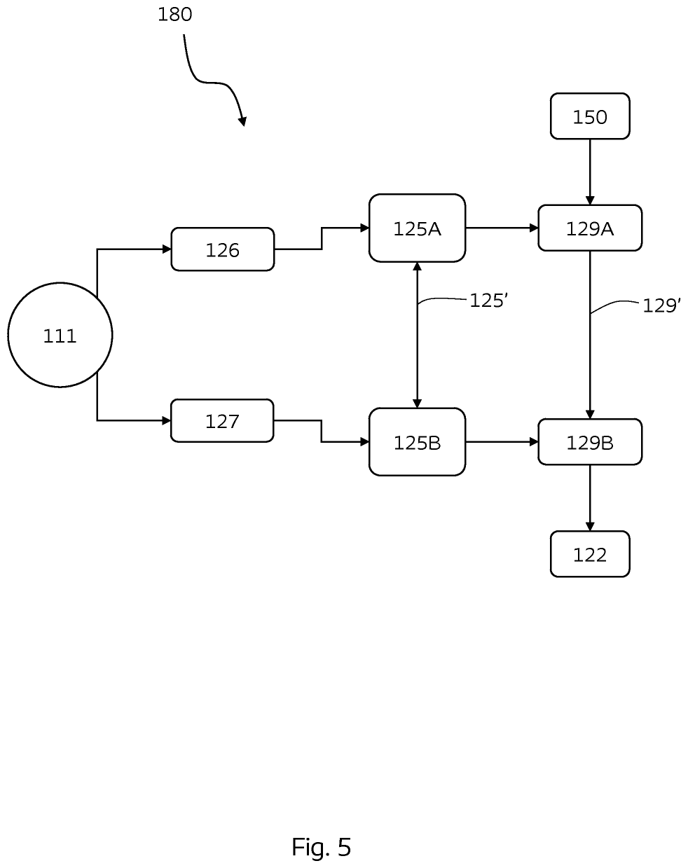

FIG. 4 illustrates a method 1000 of controlling an irrigation procedure in an irrigation system according to embodiments of the invention. The method 1000 is performed by the at least one processor, such as the processor 125 of FIG. 2 or processors 125A and 125B of FIG. 5. The reservoir of the irrigation system comprises an irrigation liquid. For example, prior to execution of the method 1000, a user has prepared the irrigation system by providing the irrigation liquid in the reservoir.

The method 1000 may comprise the steps of:
turning on 1002 the pump;
initiating 1004 the irrigation procedure;
monitoring 1006 agreement between the absolute pressure and the relative pressure; and
in accordance with the absolute pressure and the relative pressure being in agreement (P1=P2), continue the irrigation procedure; or
in accordance with the absolute pressure and the relative pressure being in disagreement (P1≠P2), terminate 1008 the irrigation procedure.

The method may comprise the optional initial step (dashed box) of taring 1001 the absolute pressure sensor and the relative pressure sensor according to previously described embodiments, such as by a method based on the description relating to FIG. 3.

The step of turning on 1002 the pump (or taring 1001 the sensors, if such step is included) may be performed by the processor in response to receiving a user input, e.g., via a user interface, indicative of such intended action. Thus, the step of turning on 1002 the pump may be initiated by the processor in response to receiving an input, such as from an on/off button.

The step of initiating 1004 the irrigation procedure may comprise to initiate pressurization of the reservoir. In embodiments, the step of initiating the irrigation procedure may be contained in the step of turning on 1002 the pump in cases where such turning on of the pump results in immediate pressurization.

The step of monitoring 1006 agreement between the absolute pressure and the relative pressure comprises to determine whether the absolute pressure and the relative pressure are in agreement or disagreement, as described in embodiments of the first aspect of the invention. The step of monitoring 1006 agreement may comprise two outcomes:
1. If the absolute pressure and the relative pressure are in agreement (P1=P2), the processor may continue monitoring 1004 agreement; and
2. If the absolute pressure and the relative pressure are in disagreement (P1≠P2), the processor may terminate 1008 the irrigation procedure, which may comprise to disable the irrigation system as such.

The nomenclature P1 and P2 used in the figure relates to the embodiment of determining (dis-) agreement described in relation to FIG. 3, but it is appreciated that (dis-) agreement may be determined by means of other methods/algorithms. As such, the nomenclature is included merely to illustrate the outcomes of the step of monitoring 1006 agreement.

It is appreciated that embodiments of the first aspect of the invention may be converted into steps of a method as described herein.

FIG. 5 illustrates a logic system 180 for use with an irrigation system 100 according to embodiments of the invention. The logic system 180 comprises the absolute pressure sensor 126 in communication with the reservoir 111, the relative pressure sensor 127 in communication with the reservoir 111, and a first processor 125A coupled to the absolute pressure sensor 126 and a second processor 125B coupled to the relative pressure sensor 127. Thus, the at least one processor 125 as referred to in previous embodiments and illustrated in FIG. 2 may comprise at least two processors including the first processor 125A and the second processor 125B.

The first processor 125A and the second processor 126B are coupled via a digital signal line 125′. The first processor 125A is coupled to a first gate 129A and the second processor 125B is coupled to a second gate 129B. The first gate 129A and the second gate 129B are coupled via a logic line 129′. The first gate 129A is coupled to the power unit 150. The second gate 129B is coupled to the pump 122. Thus, the pump 122 receives operating voltage from the power unit 150 via the gates 129A, 129B, such that the gates are required to be in a ON-state for the pump 122 to function. Thereby, through adequate operation of the gates 129A, 129B, the pump 122 may be turned off in immediate response to a change to the logic input to the gates. In a preferred embodiment, the gates are AND-transistors.

The first processor 125A and the second processor 125B are configured to output a binary signal to the respective gate 129A, 129B via respective logic lines, such that the gates may switch in accordance with the received binary signal and thus control the operation of the pump 122, which further results in a control of the irrigation procedure as such.

The first processor 125A is configured to receive absolute pressure data from the absolute pressure sensor 126 and to send absolute pressure data to the second processor 125B via the digital signal line 125′. The second processor 125B is configured to receive relative pressure data from the relative pressure sensor 127 and to send relative pressure data to the first processor 125A via the digital signal line 125′.

The first processor 125A is configured to terminate the irrigation procedure (e.g., by means of issuing an adequate binary signal prompting the first gate 129A to block the operating voltage between the power unit 150 and the pump 122), in response to any of the two scenarios:
i) if the first processor 125A does not receive relative pressure data from the second processor 125B; and/or
ii) if disagreement exists between the absolute pressure data and the relative pressure data as received from the second processor 125B.

In the first scenario, the second processor 125B may be malfunctioning and in the second scenario, disagreement exists, which may be indicative of damage to the system 100 and/or other malfunctioning parts of the system as previously discussed.

The second processor 125B is configured to terminate the irrigation procedure (e.g., by means of issuing an adequate binary signal prompting the second gate 129B to block the operating voltage between the power unit 150 and the pump 122), in response to any of the two scenarios:
a) if the second processor 125B does not receive absolute pressure data from the first processor 125A; and/or
b) if disagreement exists between the relative pressure data and the absolute pressure data as received from the first processor 125A.

In the first scenario, the first processor 125A may be malfunctioning and in the second scenario, disagreement exists, which may be indicative of damage to the system 100 and/or other malfunctioning parts of the system as previously discussed.

Through the incorporation of the logic system 180 is provided a failsafe safety mechanism, wherein the provision of two processors acting in parallel but mutually coupled via a digital signal line 125′ provides a mutual backup in case of failure such that the irrigation procedure does not continue.

The use of the terms “first”, “second”, “third” and “fourth”, “primary”, “secondary”, “tertiary” etc. does not imply any particular order but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the words “comprising” and “including” do not necessarily exclude the presence of other elements or steps than those listed.

It is to be noted that the words “a” or “an” preceding an element or method step do not exclude the presence of a plurality of such elements or method steps.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, and systems described herein are described in the general context of method steps processes or actions by a system or processor, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Although particular features have been shown and described, it will be understood that they are not intended to limit the claimed invention, and it will be made obvious to those skilled in the art that various changes and modifications may be made without departing from the spirit and scope of the claimed invention. Accordingly, the specification and drawings are to be regarded in an illustrative rather than restrictive sense. The claimed invention is intended to cover all alternatives, modifications, and equivalents.

1. An irrigation system for running an irrigation procedure for irrigation of the bowels of a user, the system comprising:
a housing comprising a reservoir for containing an irrigation liquid, and an electronics compartment sealed from the reservoir;
a tubing providing fluid communication between the reservoir and a catheter for use with the irrigation system;
a control unit comprising at least one processor;
a pump for pressurizing the reservoir to facilitate a flow of irrigation liquid from the reservoir, through the tubing, to the catheter, the pump being controllable by the at least one processor;
an absolute pressure sensor in communication with the at least one processor and configured to measure the absolute pressure in the reservoir; and
a relative pressure sensor in communication with the at least one processor and configured to measure the relative pressure between the reservoir and the electronics compartment;
wherein the at least one processor is configured to control the irrigation procedure in accordance with the absolute pressure and the relative pressure.

2. The irrigation system according to claim 1, wherein the processor is configured to monitor agreement between the absolute pressure and the relative pressure during the irrigation procedure.

3. The irrigation system according to claim 2, wherein the processor is configured to terminate the irrigation procedure in accordance with the absolute pressure and the relative pressure being in disagreement.

4. The irrigation system according to any of claims 2-3, wherein the processor is configured to disable the irrigation system in response to a disagreement between the absolute pressure and the relative pressure.

5. The irrigation system according to any of claims 1-4, wherein the absolute pressure sensor and the relative pressure sensor are configured to be tared.

6. The irrigation system according to claim 5, wherein the absolute pressure sensor and the relative pressure sensor are configured to be tared in response to an initiation of the irrigation procedure.

7. The irrigation system according to any of claims 1-6, wherein the control unit comprises a user interface.

8. The irrigation system according to claim 7, wherein the processor is configured to monitor agreement between the absolute pressure and the relative pressure during the irrigation procedure and wherein the processor is configured to communicate an error state via the user interface in response to a disagreement between the absolute pressure and the relative pressure.

9. The irrigation system according to any of claims 1-8, wherein the electronics compartment comprises at least parts of the pump, at least parts of the control unit, and at least parts of a powering circuitry configured to power the irrigation system.

10. The irrigation system according to any of claims 1-9, wherein the electronics compartment is vented relative to surroundings.

11. The irrigation system according to any of claims 1-10, wherein the control unit comprises at least two processors including a first processor connected to the absolute pressure sensor and a second processor connected to the relative pressure sensor, the first processor and the second processor further being interconnected by at least one digital signal line.

12. The irrigation system according to claim 11, wherein the first processor is configured to receive absolute pressure data from the absolute pressure sensor and to send absolute pressure data to the second processor via the digital signal line and wherein the second processor is configured to receive relative pressure data from the relative pressure sensor and to send relative pressure data to the first processor via the digital signal line.

13. The irrigation system according to claim 12, wherein the first processor is configured to terminate the irrigation procedure in response to (i) the first processor not receiving relative pressure data from the second processor and/or (ii) disagreement between the absolute pressure data and the relative pressure data as received from the second processor, and wherein the second processor is configured to terminate the irrigation procedure in response to (a) the second processor not receiving absolute pressure data from the first processor and/or (b) disagreement between the relative pressure data and the absolute pressure data as received from the first processor.

14. The irrigation system according to any of claims 11-13, wherein the first processor is connected to a first transistor via a first logic line and the second processor is connected to a second transistor via a second logic line, the first transistor further connected to a voltage source via a first voltage line and the second transistor further connected to the first transistor via a second voltage line and to the pump via a third voltage line.

15. The irrigation system according to claim 14, wherein the first transistor and the second transistor are configured as AND-gates.

16. The irrigation system according to any of claims 1-15, wherein to control the irrigation procedure comprises to control the pump.

17. A method of controlling an irrigation procedure in an irrigation system according to any of claims 1-16, the method being performed by the at least one processor of the control unit, the reservoir of the irrigation system comprising an irrigation liquid, the method comprising the steps of:
turning on the pump;
initiating the irrigation procedure;
monitoring agreement between the absolute pressure and the relative pressure; and
in accordance with the absolute pressure and the relative pressure being in agreement, continue the irrigation procedure; or
in accordance with the absolute pressure and the relative pressure being in disagreement, terminate the irrigation procedure.

18. The method according to claim 16, wherein, prior to turning on the pump, the method comprises the step of taring the absolute pressure sensor and the relative pressure sensor.
